(19) United States
(12) Patent Application Publication
(10) Pub. No.: US 2009/0214678 A1
(43) Pub. Date: Aug. 27, 2009

(54) Herbal Formulation for the Prevention and Management of Diabetes Mellitus and Diabetic Micro-Vascular Complications

(75) Inventors: Govind Prasad Dubey, Varanasi (IN); Aruna Agarwal, Varanasi (IN); Neers Vyas, New Delhi (IN); Victor G. Rajamanickam, Thanjavur (IN)

Correspondence Address: THE WEBB LAW FIRM, P.C., 700 KOPPERS BUILDING, 436 SEVENTH AVENUE, PITTSBURGH, PA, 15219, US

(73) Assignee: Govind Prasad Dubey, Varanasi (IN)

(21) Appl. No.: 11/994,362

(22) PCT Filed: Jun. 30, 2001
(86) PCT No.: PCT/IN05/00223

§ 371 Date: Jul. 21, 2008

(30) Foreign Application Priority Data
May 30, 2005 (IN) 1397/DEL/05

(51) Int. Cl. A61K 36/00; A61P 3/10

(52) U.S. Cl. 424/725

(57) ABSTRACT

A novel herbal formulation for the prevention and management of diabetes mellitus and its significance in the prevention of diabetic micro-vascular complications. A novel herbal formulation for the prevention of endothelial dysfunction Diabetes Mellitus and Diabetic Micro-Vascular complications comprising: Salacia chinensis and at least one other active constituent selected from Coccinia indica and Hippophae rhamnoides and optionally additives in trace amounts.

### FIELD OF INVENTION

This invention relates to a novel herbal formulation for the prevention of Diabetis Mellitus and Diabetic Micro-Vascular Complications. The herbal formulation may also be advantageously employed for endothelial dysfunction.

### BACKGROUND OF INVENTION

Pre-diabetes is a serious medical condition which is modifiable. Recently, Diabetes Prevention Program Study has shown that patients with pre-diabetes can prevent the development of type II diabetes by a proper treatment, which can even reduce the blood glucose levels to the normal range. A number of studies have indicated that patients with pre-diabetes can prevent or delay the development of type II diabetes by up to 68 percent through changes to their lifestyle. Normal blood glucose is below 140 mg/dl two hours after the drink. In pre-diabetes the 2-hour blood glucose is 140 to 199 mg/dl and if the 2-hour blood glucose rises to 200 mg/dl or above, a patient has diabetes.

For adults who are younger than 45 and over weight, along with other risk factors for diabetes or pre-diabetes like high blood pressure, low HDL cholesterol and high triglycerides, a family history of diabetes, a history of gestational diabetes etc are at high risk for diabetes. In fact, symptoms such as unusual thirst, frequent desire to urinate, blurred vision or a feeling of being tired most of the time for no apparent reason are often not recognized by patients. Such patients are more susceptible to coronary and peripheral artery disease and also show abnormal lipid profile.

Diabetes Control and Complications Trial (DCCT) has provided considerable information regarding the molecular mechanism, prevention and control of diabetic complications among those individuals who are suffering from different type of diabetes.

Diabetes Control and Complications Trial emerged after a comprehensive study to establish the scientific connection between the level of diabetic control and the risk of angiopathic complications in mass. The diabetic complications are mainly based on uncontrolled management of diabetes mellitus. Microangiopathic changes results in microalbuminuria, degenerative changes in micro vessels, abnormality into lipid metabolism etc. ultimately leads to gross renal, cardiac or retinopathic consequences.

Recent epidemiological studies have shown that due to varying reasons the incidence of diabetes mellitus is increasing. In one of the WHO reports the Indians are more prone to develop hypertriglyceridemia and diabetes mellitus in comparison to western countries. Diabetes is world wide in distribution and the incidence of both types of primary diabetes i.e. insulin dependent diabetes (IDDM) and non-insulin dependent diabetes (NIDDM) is increasing due to urban way of life style. However, the prevalence of both type varies considerably in different parts of the world. This seems to be due to the differences in both genetic and environmental factors. Different epidemiological studies have demonstrated that prevalence of non-insulin dependent diabetes is many times more than the insulin dependent diabetes. Though the precise etiology of diabetes is still uncertain, the environmental factors interact with a genetic susceptibility to determine which of those with a genetic predisposition actually develop the clinical syndrome and the timings of its onset. In addition to genetic, autoimmune factor also play a vital role in the onset of diabetes.

Referring to the application for endothelial dysfunction, the use of an extract of salacia oblonga is not known. However, Patricia M. Heacock et. al. has disclosed in the journal of American Dietetie Association, January 2005, the use of salacia oblonga extract as a herbal X-glucosidase inhibitor are post prandial glycemic, insulinemic in patients. Results on use of such an extract reflected that only a dosage of 1000 mg salacia oblonga reduced the plasma, glucose and serum insulin incremental areas. The other doses of S oblonga extract did not impact glycemia or insulinemia.

Further, such a paper did not teach the application of S oblonga extract with respect to endothelial dysfunction.

### OBJECTS OF INVENTION

An object of this invention is to propose a novel herbal formulation for the prevention of Diatetic Mellitus and other complications caused by Diabetes and for endothelial dysfunction.

Another object of this invention is to propose a novel herbal formulation which would delay or stop the use of insulin in patients suffering from type II diabetes.

Further object of this invention is to propose a novel herbal formulation which minimizes endothelial dysfunction in pre-diabetic and also diabetic cases.

Still further object of this invention is to propose a novel herbal formulation which regulates the abnormal lipo protein metabolism among the diabetics patients.

### STATEMENT OF THE INVENTION

According to this invention there is provided a novel herbal formulation for the prevention of endothelial dysfunction, and Diabetes Mellitus and Diabetic Micro-Vascular Complications comprising
Salacia chinensis atleast one other active constituent selected from
Coccinia indica and
Hippophae rhamnoides
and optionally additives in trace amounts

Further, according to this invention there is also provided a process of making a novel herbal formulation comprising mixing an organic extract of Salacia chinensis and atleast one of the active constituent in selected from
Hippophae rhamnoides
Coccinia indica
and optionally additives in trace amounts.

### DETAILED DESCRIPTION OF THE PRESENT INVENTION

Coccinia indica is a creeping plant found in entire India particularly in the Tribal locality of Vindhya and Satpura hills. It has three species namely Coccinia grandis, Coccinia indica and Coccinia cordifolia. The fruits of the plant is essentials used for a vegetable. It contain β-sitosterol and taraxerol from fruits; seeds are rich in essential fatty acid like palmitic acid, oleic acid and linoleic acids. The use of Coccinia indica as a herbal preparation for the applications described herein is not hitherto known.

Hippophae rhamnoides:

Hippophae rhamnoides belongs to the family of ‘Elaegnaceae’, is a small genus of shrubs and trees and in native of temperate region. It contains vitamins such as carotene, flavonoides, folic acid, fatty acids, and tannic acid. The ripe fruits contain many typy of vitamins and stem contains 5-HT also. The use of Hippophae rhamnoides as a herbal preparation for the applications described herein is not hitherto known.

Salacia chinensis:

Salacia is a small deciduous plant found in eastern and western Peninsula, Meghalaya and in the central India particularly near the bank of Narmada valley up to the 3000 feet from sea level. The length of leaves varies from 3 to 6 inches and about 1 to 2 inches. The flowering starts in the month of December and January, and fruits appears in the month of April. The bark of root is golden colour and after cutting the root, seven wheel appears and it has a variety of colours. This plant has have a 3 sub species namely Salacia chinensis, Salacia latifolia and Salacia oblonga. It is also known as Salacia prinoides. The root is utilized by the local people for the treatment of inflammatory process and also for liver disorders and for the management of polyurea and polydyspepsia. The use of Salacia chinensis as a herbal preparation for the applications described herein is not hitherto known.

The novel formulation of the present invention comprises the following constituents:
1. Coccinia indica (Bimbl)
2. Hippophae rhamnoides (Badriphal)
3. Salacia chinensis (Sapta chakra)
and present in the range of:
1. Coccinia indica—200-400 mg.
2. Hippophae rhamnoides—200-400 mg.
3. Salacia chinensis—350-550 mg.
for every 1000 mg of said formulations.
also preferably the formulation comprises:
1. Coccinia indica—200-300 mg.
2. Hippophae rhamnoides—250-400 mg.
3. Salacia chinensis—400-500 mg.
for every 1000 mg of the preparation.

The formulation may also comprise known additives such as mineral, vitamin, salts, fillers (for encapsulation) and binders, if required and present in trace amounts.

Extraction Procedure of Salacia chinensis:

Root of Salacia chinensis is used for the hydro-alcoholic extraction. Extraction was done through hot percolation method by soxhlet apparatus using water and alcohol as a solvent. Extraction was carried out continuously for 70 hrs. at boiling point of the solvent used. After 70 hrs, maintaining the same temperature solvent used in the extraction was recovered to a possible amount through single distillation. The obtained hydro-alcoholic extract was solidified to a pasty mass over thermostatic water bath at a maintained temperature as above. The yield was calculated after weighing the extracted quantity.

Quantitative Estimation of Alcoholic Extract of Salacia chinensis for the Separation of Active Chemical Constituents of Salacia chinensis by Chromatographic Procedures.

After extraction of organic extract of Salacia chinensis, a chromatographic separation was carried out by using thin layer column and high performance liquid chromatography. Various chemical compounds like leucopelargonidin, tetramer, dulcitol and gutta were separated and characterized on the basis of different results obtained by IR, UV, NMR for proton, NMR for carbon, Mass and elemental analysis.

Extraction Procedure of Hippophae rhamnoides and Coccinia indica:

The dried fruits of Hippophae rhamnoides was extracted with hydro-alcoholic solvent (70:30) at the temperature of 60 to 70 continuously for 70 hrs. After a detailed chemical characterization the extracted material was taken for assessment of therapeutic potentials. The same extraction procedure was adopted to obtain the yield of the plant Coccinia indica for purpose of preparation of the formulation.

After processing the yield with column chromatography of the plants under investigation HPTLC and spectroscopic method the extract were taken for the assessment of their safety and efficacy profile.

The herbal formulation of the present invention was obtained by mixing the organic extract of Salacia chinensis with the extract of Hippophae rhamnoides and Coccinia indica.

The extract of Salacia oblonga when taken twice (1000 mg in two equal doses) showed reduction in blood glucose level in comparison to control series but Salacia chinensis showed better effect on blood glucose level in comparison to salacia oblonga. Similarly, when diabetics is caused in rats using streptozotocin a far better result is obtained after 12 weeks with Salacia chinensis compared to salacia oblonga as salacia chinensis showed contant decline in blood glucose level as clearly shown in Table 1.

TABLE 1
Effect of Salacia chinensis and Salacia oblonga on Blood, glucose level
among Streptozotocin treated rats - a comprehensive study

| | Blood Glucose level (mg %) | | | |
|---|---|---|---|---|
| Experimental | | After | After | After |
| Groups | Initial | 7 days | 6 weeks | 12 weeks |
| Normal control | 63.97 ± 3.42 | 68.44 ± 5.02 | 67.01 ± 4.82 | 64.73 ± 5.08 |
| (N = 10) | | | | |
| Treated with | 67.11 ± 3.97 | 192.84 ± 11.64 | 176.94 ± 7.72 | 164.80 ± 8.22 |
| Streptozotocin | | | | |
| (N = 10)* | | | | |
| Streptozotocin + | 64.72 ± 6.84 | 176.01 ± 6.42 | 144.52 ± 5.11 | 102.98 ± 4.28 |
| Salacia chinensis | | | | |
| (N = 10)** | | | | |
| Streptozotocin + | 67.94 ± 5.83 | 182.45 ± 9.25 | 161.02 ± 5.85 | 123.44 ± 6.02 |
| Salacia oblonga | | | | |
| (N = 10)*** | | | | |

Comparison:

| | | | | |
|---|---|---|---|---|
| *vs** | p > 0.05 | p < 0.01 | p < 0.001 | p < 0.001 |
| *vs*** | p > 0.05 | p < 0.05 | p < 0.001 | p < 0.001 |
| *vs*** | p > 0.05 | p < 0.05 | p < 0.001 | p < 0.001 |

The results of table 2 initially, reflects that when organic extract of Salacia chinensis, Hippophae rhamnoides and Coccinia indica was given simultaneously the decrease in blood glucose level is less in animals compared to the results obtained by administrating salacia chinensis alone. Whereas, table 2 also shows that in the long run say after 3 months the combined effect of Salacia chinensis, Hippophae rhamnoides and Coccinia indica give much better results. Thus, the herbal formulation reduces the blood glucose level to a great extent both for fasting and post prandial PP. Table 2:

TABLE 2
Clinical study
Effect of Salacia chinensis and novel test formulation in blood glucose
level among pre diabetes cases.

| | | | | | Comparison | |
|---|---|---|---|---|---|---|
| | | | | | Initial | Initial |
| | Blood glucose level (mg %) | | | | Vs | Vs |
| Treatment | Initial | | After 3 months | | After | After |
| groups | Fasting | PP | Fasting | PP | 3 months | 3 months |
| Control | 118.45 ± 6.25 | 193.74 ± 8.92 | 124.29 ± 6.43 | 188.72 ± 9.54 | P < 0.001 | P < 0.05 |
| Groups | | | | | | |
| (only dietary | | | | | | |
| control) | | | | | | |
| (N = 48) | | | | | | |
| Treated | 121.73 ± 9.42 | 197.88 ± 11.64 | 104.22 ± 5.28 | 157.34 ± 4.84 | P < 0.001 | P < 0.001 |
| Group | | | | | | |
| Dietary | | | | | | |
| control + | | | | | | |
| Salacia | | | | | | |
| chinensis | | | | | | |
| (N = 53) | | | | | | |
| Treated | 119.48 ± 8.64 | 201.94 ± 10.40 | 98.02 ± 4.62 | 145.35 ± 6.25 | P < 0.001 | P < 0.001 |
| group dietary | | | | | | |
| control + | | | | | | |
| test | | | | | | |
| formulation | | | | | | |
| organic | | | | | | |
| extract of | | | | | | |
| Salacia | | | | | | |
| chinensis & | | | | | | |
| Hippophae | | | | | | |
| Rhamnoides & | | | | | | |
| Coccinia | | | | | | |
| indica | | | | | | |
| (N = 61) | | | | | | |

Table 3 shows that the combined effect of the three ingredients of the herbal formulation gives a better result in rats which are treated with streptozotocin. After a duration of 12 weeks it is seen that blood glucose level decreases when streptozotocin treated rats are given the dose of herbal formulation comprising Salacia chinensis, Hippophae rhamnoides and Coccinia indica.

TABLE 3
Effect of organic extract of Salacia chinensis, Hippophae rhamnoides
and Coccinia indica on blood glucose level in Streptozotocin treated
experimental rats.

| | Blood Glucose level (mg %) | | |
|---|---|---|---|
| Experimental | | After | After |
| group | Initial | 6 weeks | 12 weeks |
| Normal | 63.97 ± 3.42 | 67.01 ± 4.82 | 64.73 ± 5.08 |
| control | | | |
| (N = 10)* | | | |
| Streptozotocin | 67.11 ± 3.92 | 176.94 ± 7.72 | 164.80 ± 8.22 |
| treated | | | |
| (N = 10)** | | | |
| Streptozotocin + | 61.45 ± 4.52 | 158.84 ± 9.02 | 94.02 ± 5.11 |
| Salacia | | | |
| chinensis & | | | |
| Hippophae | | | |
| rhamnoides & | | | |
| Coccinia | | | |
| indica | | | |
| (N = 10)*** | | | |

Comparison:

| | | | |
|---|---|---|---|
| *vs** | p > 0.05 | p > 0.001 | p < 0.001 |
| *vs*** | p > 0.05 | p > 0.001 | p < 0.001 |
| **vs*** | p < 0.05 | p > 0.001 | p < 0.001 |

The marked increase in the level of C-reaction protein (CRP), homocystin and interleukin 6 in diabetes mellitus type II causes endothelial dysfunction.

This endothelial dysfunction is regulated to great extent by the use of herbal formulation (containing organic extract of Salacia chinensis, Hippophae rhamnoides & Coccinia indica), as shown in Table 4:

TABLE 4
Endothelial dysfunction and its regulation by a herbal formulation
among, diabetes mellitus type II cases

| | | | After | After | After | After | After |
|---|---|---|---|---|---|---|---|
| | | | 6 | 12 | 18 | 24 | 30 |
| parameters | Group | Initial | week | weeks | weeks | weeks | week |
| CRP | Test | 3.22 ± 1.72 | 2.05 ± 1.82 | 1.35 ± 0.85 | 1.35 ± 0.38 | 1.48 ± 0.47 | 1.28 ± 0.35 |
| (mg/lit.) | drug | | | | | | |
| | treated | | | | | | |
| | Anti | 3.28 ± 1.02 | 3.10 ± 0.98 | 2.98 ± 0.80 | 3.06 ± 0.75 | 2.85 ± 0.68 | 2.82 ± 0.64 |
| | Diabetic | | | | | | |
| | agent | | | | | | |
| | (Control) | | | | | | |
| Homocysteine | Test | 21.85 ± 5.82 | 16.42 ± 3.85 | 17.2 ± 4.28 | 12.80 ± 4.75 | 0.5 ± 3.9 | 8.63 ± 4.6 |
| (μmol/lit.) | drug | | | | | | |
| | treated | | | | | | |
| | Anti | 23.45 ± 2.73 | 23.21 ± 3.01 | 22.78 ± 3.04 | 23.11 ± 2.94 | 22.98 ± 2.72 | 22.94 ± 2.44 |
| | diabetic | | | | | | |
| | agent | | | | | | |
| | (Control) | | | | | | |
| Inter-leukin₆ | Test | 3.45 ± 1.75 | 2.24 ± 1.28 | 1.92 ± 0.98 | 1.70 ± 0.75 | 1.62 ± 0.78 | 1.24 ± 0.62 |
| (pg/ml) | drug | | | | | | |
| | treated | | | | | | |
| | Anti | 3.52 ± 1.03 | 3.45 ± 0.75 | 3.28 ± 0.92 | 3.46 ± 0.84 | 3.18 ± 0.65 | 3.08 ± 0.58 |
| | diabetic | | | | | | |
| | Agent | | | | | | |
| | (Control) | | | | | | |

The organic extract of Salacia chinensis along with Hippophae rhamnoides and Coccinia indica was administrated to prediabetic cases which showed significant decrease in low density lipoprotein, cholesterol and HDLC compared to the organic extract of Salacia chinensis alone. Table 5 & 5(a) clearly reflects that the combined effect of herbal formulation of the present invention gives much better results.

TABLE 5
Effect of Salacia chinensis and Novel Test Formulation in the regulation
of LDL-c and HDL-c

| | Low density | | | | | |
|---|---|---|---|---|---|---|
| | Lipoprotein | | Comparison | HDL Cholesterol | | Comparison |
| | (mg/dl) | | Initial vs | (mg/dl) | | Initial vs |
| Treatment | | After 3 | After 3 | | After 3 | After 3 |
| group | Initial | months | months | Initial | months | months |
| Control | 123.84 ± 6.23 | 118.56 ± 5.95 | P < 0.001 | 47.95 ± 2.82 | 45.88 ± 3.02 | P < 0.01 |
| group | | | | | | |
| (only | | | | | | |
| dietary | | | | | | |
| control) | | | | | | |
| (N = 48) | | | | | | |
| Treated | 125.65 ± 7.01 | 110.85 ± 5.35 | P < 0.001 | 46.28 ± 2.11 | 48.01 ± 3.11 | P < 0.01 |
| Salacia | | | | | | |
| chinensis | | | | | | |
| along with | | | | | | |
| dietary | | | | | | |
| control | | | | | | |
| (N = 53) | | | | | | |
| Treated | 126.38 ± 6.82 | 93.82 ± 6.02 | P < 0.001 | 44.25 ± 2.06 | 50.23 ± 4.03 | P < 0.001 |
| with Test | | | | | | |
| formulation | | | | | | |
| along with | | | | | | |
| dietary | | | | | | |
| control | | | | | | |
| (N + 61) | | | | | | |

TABLE 5a
Effect of Salacia chinensis and Novel Test Formulation in the regulation
of Total cholesterol and triglycerides

| | Total cholesterol | | Comparison | Triglycerides | | Comparison |
|---|---|---|---|---|---|---|
| | (mg/dl) | | Initial vs | (mg/dl) | | Initial vs |
| Treatment | | After 3 | After 3 | | After 3 | After 3 |
| group | Initial | months | months | Initial | months | months |
| Control | 216.84 ± 8.97 | 201.72 ± 6.82 | P < 0.001 | 224.87 ± 8.25 | 208.86 ± 9.25 | P < 0.001 |
| group | | | | | | |
| (only | | | | | | |
| dietary | | | | | | |
| control) | | | | | | |
| (N = 48) | | | | | | |
| Treated | 221.88 ± 10.02 | 197.45 ± 8.52 | P < 0.001 | 230.26 ± 8.25 | 213.87 ± 9.26 | P < 0.001 |
| Salacia | | | | | | |
| chinensis | | | | | | |
| along with | | | | | | |
| dietary | | | | | | |
| control | | | | | | |
| (N = 53) | | | | | | |
| Treated | 227.43 ± 10.55 | 182.25 ± 7.34 | P < 0.001 | 229.85 ± 10.30 | 172.24 ± 8.04 | P < 0.001 |
| with Test | | | | | | |
| formulation | | | | | | |
| along with | | | | | | |
| dietary | | | | | | |
| control | | | | | | |
| (N + 61) | | | | | | |

When the organic extract of Coccinia indica, Hippophae rhamnoides, Salacia chinensis was orally administered the most effective result were obtained. A significant regulation of blood glucose levels, modification of abnormal lipid metabolism along with triglycerides and improvement in psychological complaints were recorded under influence of this new herbal formulation. A general feeling of well being and improvement in mental and physical stress of the subjects was noticed and thus the subject's work performance improved significantly as shown in Table 6.

TABLE 6
Effect of organic extract of Salacia chinensis and test formulation on clinical
Symptomatology in Pre-diabetic cases

| | Clinical Symptomatology (Improvement in percent) | | | | | | | | | |
|---|---|---|---|---|---|---|---|---|---|---|
| | Neuro logical | | | | | | | | [symbol] feeling of | |
| | complications | | Early fatigue | | Anxiety | | Depression | | well being | |
| | | After | | After | | After | | After | | After |
| Treatment | Before | treatment | Before | treatment | Before | treatment | Before | treatment | Before | treatment |
| Group | treatment | (12 wks) | treatment | (12 wks) | treatment | (12 wks) | treatment | (12 wks) | treatment | (12 wks) |
| Control group | 23 | 25 | 41 | 38 | 36 | 37 | 33 | 34 | 46 | 45 |
| (N = 48) | (47.91%) | (52.08%) | (85.54%) | (79.16%) | (75%) | (77.08%) | (68.75%) | (70.83%) | (95.83%) | (93.75%) |
| Treated with | 41 | 22 | 50 | 28 | 47 | 24 | 38 | 21 | 48 | 28 |
| Salacia | (77.35%) | (41.50%) | (94.33%) | (52.83%) | (88.67%) | (45.28%) | (71.69%) | (39.62%) | (90.56%) | (52.83%) |
| chinensis | | | | | | | | | | |
| (N = 53) | | | | | | | | | | |
| Treated with | 53 | 19 | 59 | 18 | 57 | 21 | 46 | 19 | 58 | 11 |
| test | (86.88%) | (31.14%) | (96.72%) | (29.50%) | (93.44%) | (34.42%) | (75.40%) | (31.11%) | (95.08%) | (18.03%) |
| formulation | | | | | | | | | | |
| (N = 61) | | | | | | | | | | |

[symbol]

indicates data missing or illegible when filed

It was found that when the organic extract of Coccinia indica along with the organic extract of Hippophae rhamnoides was given orally in the prescribed doses continuously for long period the subject showing evidence of potential diabetes mellitus exhibited reduction in fasting and post prandial blood glucose levels. A decreased blood pressure levels were also recorded among those cases showing mild to moderate elevation of systolic and diastolic blood pressure. The formulation of the present invention regulates the blood glucose levels, blood pressure levels, and abnormal lipo-proteins including triglycerides. When the organic extract of Coccinia indica along with the organic extract of dry fruits of Hippophae rhamnoides and the organic extract of Salacia chinensis was administered in a specific determine effective doses better results were obtained when the values of blood glucose levels were measured, both fasting and post prandial blood glucose levels reduced and regulated in more effective manner than the earlier group of study. It has been concluded that the formulation has potentiality to prevent the pre-diabetics from entering in to type II diabetes.

### EXAMPLES

### Example 1

The organic extract of Salacia chinensis in the dose of 25-30 mg was administered did not show any change-in blood glucose level among alloxan and streptozotocin induced experimental rats.

### Example 2

When the organic extract of Salacia chinensis was administered in the dose of 70-100 mg to hyperglycaemic experimental rats, 40 percent of reduction in high blood glucose level was noticed.

### Example 3

When the organic extract of leaves of Coccinia indica in the dose of 10-20 mg was introduced to alloxan and streptozotocin induced hyperglycaemic rats no hypoglycaemic activity could be noticed. The organic extract of the leaves in the dose of 60-80 mg was orally administered exerted beneficial effect on blood glucose level.

### Example 4

When the organic extract of Salacia chinensis in the dose of 40-60 mg and Coccinia indica in the dose of 30-50 mg was given to hyperglycaemic rats a significant reduction in blood glucose level was noticed.

### Example 5

In this experimental group the organic extract of Salacia chinensis in the dose of 50-75 mg along with Hippophae rhamnoides in the dose of 30-50 mg was given orally to experimentally induced hyperglycaemic rats a significant fall in blood glucose level was recorded.

### Example 6

The organic extract of Salacia chinensis in the dose of 30-50 mg, Hippophae rhamnoides in the dose of 25-35 mg and Coccinia indica in the dose of 2-35 mg was given to experimental hyperglycaemic rats maximum beneficial effects were noticed on blood glucose level and also in regulating the kidney function

### Example 7

In another group of clinical study when the organic extract of Salacia chinensis administered in the dose of 350-500 mg per day along with the organic extract of Hippophae rhamnoides in the dose of 250-350 mg per day improvement in neurological complaints were noticed along with hypoglycaemic effects.

### Example 8

When the organic extract of Salacia chinensis in the dose of 400-500 mg per day along with the organic extract of Hippophae rhamnoides in the dose of 200-350 mg per day was introduced a significant decrease in blood pressure was noticed among the pre-diabetic cases. A general body immunity also enhanced due to reduced oxidative injury and modification in abnormal immune complex phenomena.

### Example 9

When the organic extract of Coccinia indica in the dose of 250-300 mg per day was given along with the organic extract of Salacia chinensis in the dose of 400-550 mg per day, a better results of both fasting and post prandial glucose levels were noticed. A general feeling of well being was also reported by the diabetic patients.

### Example 10

When the organic extract of Hippophae rhamnoides in the dose of 300-500 mg and Coccinia indica in the dose of 200-250 mg, was given a general feeling of well being along with improvement in anxiety level were noticed in pre-diabetes cases and type II diabetes also.

### Example 11

When the organic extract of Coccinia indica in the dose of 200-300 mg per day was given along with the organic extract of hippophae rhamnoides in the dose of 250-450 mg per day, a significant reduction in total cholesterol and triglycerides were noticed among the diabetes mellitus cases who showed hypercholesterolemia and hypertrigly ceridemia.

### Example 12

The cases of pre-diabetes receiving the organic extract of Coccinia indica in the dose of 250-350 mg per day along with the organic extract of Salacia chinensis in the dose of 350-400 mg per day was introduced, significant reduction in Low-density lipoprotein cholesterol and mild increase in high density lipoprotein cholesterol was noticed along with decrease in blood glucose level.

### Example 13

When the organic extract of Coccinia indica in the dose of 200-300 mg per day along with the organic extract of Hippophae rhamnoides in the dose of 250-400 mg per day, organic extract of Salacia chinensis in the dose of 400-650 mg per day was administered among diabetes mellitus cases significant improvement in fasting and post prandial blood glucose levels were observed along with regulation of TC, LDL-c and triglycerides. Subjects also reported a general feeling of well being and better mental performance.

1-7. (canceled)

8: A novel herbal formulation for the prevention of endothelial dysfunction Diabetes Mellitus and Diabetic Micro-Vascular complications comprising:
350-550 mg Salacia chinensis and at least one other active constituent selected from 200-400 mg Coccinia indica and 200-400 mg Hippophae rhamnoides.

9: A novel herbal formulation for the prevention of endothelial dysfunction Diabetes Mellitus and Diabetic Micro-Vascular complications comprising:
350-550 mg Salacia chinensis,
200-300 mg Coccinia indica, and
200-400 mg Hippophae rhamnoides.

10: A novel herbal formulation for the prevention of endothelial dysfunction Diabetes Mellitus and Diabetic Micro-Vascular complications comprising:
200-300 mg Coccinia indica,
250-400 mg Hippophae rhamnoides, and
400-500 mg Salacia chinensis
for every 1000 mg of the formulation.

11: The novel herbal formulation as claimed in claim 8, wherein additional additives comprise one or more compounds or compositions selected from the group consisting of minerals, vitamins, salts and binders.

12: A process of making a novel herbal formulation comprising mixing an organic extract of Salacia chinensis with the extract of at least one of the active constituents selected from Hippophae rhamnoides and Coccinia indica.

13: A process of making a novel herbal formulation comprising mixing an organic extract of Salacia chinensis, Hippophae rhamnoides and Coccinia indica with at least one additive selected from the group consisting of minerals, vitamins, salts and binders.
